(19) United States
(12) Patent Application Publication
(10) Pub. No.: US 2007/0071759 A1
(43) Pub. Date: Mar. 29, 2007

(54) ANTIBODY-IMMUNE CELL LIGAND FUSION PROTEIN FOR CANCER THERAPY

(75) Inventors: Seung-Uon Shin, Miami, FL (US); Joseph D. Rosenblatt, Hollywood, FL (US); Khaled Tolba, Miami, FL (US)

Correspondence Address: AKERMAN SENTERFITT, P.O. BOX 3188, WEST PALM BEACH, FL, 33402-3188, US

(73) Assignee: University of Miami, Miami, FL (US)

(21) Appl. No.: 11/427,628

(22) Filed: Jun. 29, 2006

Related U.S. Application Data
(60) Provisional application No. 60/695,114, filed on Jun. 29, 2005.

(51) Int. Cl. A61K 39/395; C07H 21/04; C12P 21/06; C12N 5/06; C07K 16/30; C07K 16/46

(52) U.S. Cl. 424/155.1; 435/69.1; 435/328; 435/320.1; 530/388.8; 530/391.1; 536/23.53

(57) ABSTRACT

Compositions for treatment of cancer comprising chimeric fusion molecules that bind to an antigen on a pathogenic cell and to an immune cell. The molecules redirect the immune cells to a pathogenic cell. The purified fusion proteins demonstrated ability to bind antigen on the surface of tumor cells and cell surface receptors on immune cells such as NK cells. The chimeric fusion proteins showed increased cytotoxic activity directed against tumor targets.

### CROSS-REFERENCE TO RELATED APPLICATIONS

The present application claims the priority of U.S. Provisional Patent Application No. 60/695,114, filed Jun. 29, 2005, entitled ANTIBODY-NKG2D LIGAND FUSION PROTEIN FOR CANCER THERAPY, which is incorporated herein by reference in its entirety.

### STATEMENT REGARDING FEDERALLY SPONSORED RESEARCH OR DEVELOPMENT

The United States Government may have certain rights in this invention pursuant to U.S. Army Medical Research and Material Command (Department of Defense) Contract No. BC033005 (W81XWH-04-1-0733).

### FIELD OF THE INVENTION

The invention relates to the fields of medicine, immunology, and oncology. Specifically, the invention relates to compositions and methods for stimulating an immune system with the objective of killing cancer cells as well as stimulating immune response.

### BACKGROUND OF THE INVENTION

NKG2D ligands are inducible stress response molecules expressed on virally infected and transformed cells.¹ NKG2D ligands activate the NKG2D receptor, a C type lectin-like receptor expressed on effector cells belonging to the innate and adaptive immune systems, and offer an effective link between innate and adaptive immunity necessary to mount potent anti-tumor response.² Directed -expression of NKG2D ligands by tumors has led to tumor regression in multiple murine tumor models.^{3,4}

The anti-HER2 antibody (Herceptin) is approved for the treatment of metastatic breast cancer. However, Herceptin is effective only in a small percent of patients whose tumors express HER2. Antibody-based cancer therapy is thought to lead to tumor destruction by activation of antibody dependent cytotoxicity (ADCC) and/or through direct effects on signaling by targeted receptors such as HER2. ADCC may be a major anti-cancer mechanism and it could be more effectively elicited in the presence of activated effector cells with increased cytolytic capacity that is obtained through activation of a local innate immune response. Furthermore an enhanced local innate response may lead to more efficient priming of an adaptive T cell mediated response.

It is thus important to develop new compositions for treatment of cancer.

### SUMMARY

The invention relates to the development of tumor-targeting chimeric molecules containing both immune cell ligands and a carrier domain of anti-tumor antibody.

In the illustrative embodiments described below, chimeric molecule include: an Ig domain from an anti-HER2/neu antibody fused to Rael 1β.

In a preferred embodiment, the invention provides a pharmaceutical composition comprising a chimeric fusion molecule, wherein the chimeric fusion molecule comprises an antigen binding domain and an immune cell binding domain. Preferably, the pharmaceutical composition is used in treating cancer.

In another preferred embodiment, the antigen binding domain comprises an isolated antibody or fragments thereof. The isolated antibody or fragments thereof comprises immunoglobulin heavy and light chains and/or immunoglobulin variable and constant regions. Preferably, the isolated immunoglobulin variable region comprise Fab, Fab′, F(ab′)₂, and Fv fragments and/or immunoglobulin constant regions, C_{H}1, hinge, C_{H}2 and C_{H}3.

In another preferred embodiment, the isolated antibody or fragments thereof are fused to an immune cell binding domain. In accordance with the invention, the isolated antibody is fused to the immune cell binding domain via the immunoglobulin constant regions, C_{H}1, hinge, C_{H}2 or C_{H}3. Preferably, the isolated antibody is fused to the immune cell binding domain via the immunoglobulin constant region, C_{H}3.

In another preferred embodiment, the immune cell binding is a ligand specific for an NK cell receptor, a monocyte receptor, a B-cell surface receptor, and/or a T cell surface receptor. Preferably, the immune cell binding domain is a ligand for a natural killer cell, (NK cell), such as, for example, an NKG2D ligand and variants thereof and/or MHC class I alpha and beta chains and/or UL 16 binding proteins.

In another preferred embodiment, the UL16 binding proteins are selected from the group consisting of ULBP1, ULBP2, ULBP3, and ULBP4.

In another preferred embodiment, the chimeric fusion protein is directed to a breast cancer antigen. Examples include, HER2, human telomerase reverse transcriptase (hTERT), cytochrome P450 isoform 1B1 (CYP1B1) CA 27.29, or CA 15-3 antigen, or leukemia and/or lymphoma antigens, e.g. anti CD20, anti-CD22, or anti-CD52, or antibody sequences directed against lung or colon cancer antigens e.g. anti-EGFR, or prostate cancer antigens e.g. PSMA, as examples of alternative specificity. However, any antibody binding site specific for a tumor antigen can be used. The chimeric fusion molecule can be comprised within a pharmaceutical carrier suitable for administration to an animal subject.

In a preferred embodiment, NKG2D ligands are directly targeted to tumor cells using an antibody-NKG2D ligand fusion protein targeted against the breast tumor antigen HER2.

In another preferred embodiment, a nucleic acid expresses a chimeric fusion molecule, wherein the chimeric fusion molecule comprises a tumor antigen binding domain and an immune cell binding domain. For example, the immune cell binding domain is obtainable by polymerase chain reactions using primers SEQ ID NO's: 1 and 2. The immune cell binding domain nucleic acids are ligated to nucleic acid sequences preferably express an anti-tumor antigen binding domain.

In another preferred embodiment, a method of treating cancer in an animal subject comprises administering to the animal subject a pharmaceutical composition comprising a chimeric fusion molecule, wherein the chimeric fusion molecule comprises a tumor antigen binding domain and an immune cell binding domain. Preferably, the immune cell binding domain is a ligand specific for an NK cell receptor, a monocyte receptor, a B-cell surface receptor, and/or a T cell surface receptor. For example, the immune cell binding domain is a ligand specific for a natural killer cell receptor (NK cell). Preferably, the immune cell ligand is an NKG2D ligand and variants thereof and/or MHC class I alpha and beta chains and/or UL 16 binding proteins.

Antibody specificity can be directed to any known tumor antigens (eg Her2/neu in breast cancer, EGFR in solid tumors, CD20 in lymphoma, etc).

Unless otherwise defined, all technical terms used herein have the same meaning as commonly understood by one of ordinary skill in the art to which this invention belongs. Commonly understood definitions of molecular biology terms can be found in Rieger et al., Glossary of Genetics: Classical and Molecular, 5th edition, Springer-Verlag: New York, 1991; and Lewin, Genes V, Oxford University Press: New York, 1994.

Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions will control. In addition, the particular embodiments discussed below are illustrative only and not intended to be limiting.

### BRIEF DESCRIPTION OF THE DRAWINGS

There is shown in the drawings embodiments, which are presently preferred, it being understood, however, that the invention can be embodied in other forms without departing from the spirit or essential attributes thereof.

FIG. 1A is a schematic illustration showing the structure of Anti-HER2 IgG3-Rae-1βfusion proteins. FIG. 1B is a scan showing the SDS-PAGE analysis of anti-HER2 IgG3-Rae-1β fusion protein. The purified IgG3-Rae-1β fusion proteins were analyzed under non-reducing condition. Control anti-HER2 IgG3 is included for comparison.

FIG. 2 are histograms showing the binding analysis of anti-HER2 IgG3-Rae-1β fusion proteins. The Rae-1β moiety of anti-HER2 antibody-Rae-1β fusion proteins has been tested for binding to NKG2D on freshly isolated NK cells or murine NK cell line KY-2 cells. Anti-HER2 antibody-Rae-1β fusion proteins, CH3-Rae-1β filled with red color) and H-Rae-1β (blue colored line), showed binding ability, but anti-HER2 IgG3 (green colored line) and isotype control filled with purple color) did not bind. Both CH3-Rae-1β filled with red color) and H-Rae-1β (blue colored line) have recognized HER2 and have been detected through the Rae-1β moiety, but anti-HER2 IgG3 (green colored line) and isotype control filled with purple color) did not have been detected through the Rae-1β moiety.

FIG. 3 is a histogram showing anti-HER2 IgG3-Rae-1β fusion protein-mediated enhancement of perforin production in KY-2 NK cells. Histograms demonstrate intracellular perforin expression of IL2 (100U)-stimulated KY-2 cell cultured in the presence of anti-HER2b IgG3-CH3-Rae-1β fusion protein at the various concentrations (0.1 μg: filled with blue color, 0.5 μg: filled with orange color, 2 μg: filled with red color), anti-HER2 IgG3 (2 μg: blue colored line), and isotype control (2 μg: black colored line).

FIG. 4 is a graph showing enhancement of tumor-directed NK cell-mediated cytotoxicity by anti-HER2 IgG3-Rae-1β fusion protein. Freshly isolated NK cells were stimulated in the presence of anti-HER2 IgG3-Rae1β fusion proteins (10 μg/well, C_{H}-Rae1β; filled with red color, H-Rae-1β; filled with green color), anti-HER2 IgG3 (10 μg/well, filled with purple color), or control anti-dansyl IgG3 (10 μg/well, black line). After 2 days, NK were cocultured in round-bottom 96-well plates with the ⁵¹Cr-labeled tumor cell lines MC38-HER2 at different E:T ratios. After 5 h of incubation, chromium release was measured. The results of three different donors are presented as mean ±SE of triplicate wells.

FIG. 5 are scans of SDS-PAGE gels showing anti-HER2 IgG3-Rae-1β fusion proteins of the expected molecular weight were secreted as the fully assembled H₂L₂ form.

FIG. 6 shows histograms of binding of anti-HER2 IgG3-Rae-1β fusion proteins. Anti-HER2 IgG3-Rae-if) fusion proteins bound to HER2+on the surface of tumor cells and Rae1β fusion proteins recognized NKG2D receptor as displayed on the NKG2D-Fc (human IgG1) fusion protein or on NK cells through Rae1β moiety. Hinge-Rae1β fusion showed reduced binding to NKG2D compared to CH3-Rae-1β.

FIGS. 7A-7C are graphs showing NK cell-mediated direct lysis. Anti-HER2 IgG3 (IgG3) and effector cells exhibited little tumor-directed cytotoxicity (5-10%) using KY-2 cells as effectors at the indicated effector:target ratios. H-Rae1β fusion protein exhibited very little tumor-directed cytotoxicity (5%). Incubation of targets with CH3-Rae1β fusion markedly enhanced NK cell-mediated killing (15-59%).

FIGS. 8A and 8B are graphs showing redirected lysis with KY2. Up to 22% redirected lysis was observed when P815 or J774 cells primed with anti-HER2 IgG3-CH3-Rae1β were incubated with KY-2 cells. This lysis was greater than that seen with control anti-HER2 IgG3 (<6%).

FIG. 9 is a graph showing anti-tumor activity of anti-HER2 IgG3-CH3-Rae1β against MC38-HER2 . Anti-HER2 IgG3-Rae1β fusion proteins inhibited the growth of the murine MC38-HER2 in C57BL6 to a greater extent than PBS.

### DETAILED DESCRIPTION

A chimeric fusion molecule that targets immune cells to a tumor. The molecule is designed to be specific for any tumor antigen and can further be tailored to tumor antigens that are specific to a patient. Methods of treating cancer or any infected cell are provided.

Definitions

In accordance with the present invention and as used herein, the following terms are defined with the following meanings, unless explicitly stated otherwise.

As used herein, “a”, “an,” and “the” include plural references unless the context clearly dictates otherwise.

“Substantially purified” refers to nucleic acid molecules or proteins that are removed from their natural environment and are isolated or separated, and are at least about 60% free, preferably about 75% free, and most preferably about 90% free, from other components with which they are naturally associated.

As used herein, “cancer” refers to all types of cancer or neoplasm or malignant tumors found in mammals, including, but not limited to: leukemias, lymphomas, melanomas, carcinomas and sarcomas. Examples of cancers are cancer of the brain, breast, pancreas, cervix, colon, head & neck, kidney, lung, non-small cell lung, melanoma, mesothelioma, ovary, sarcoma, stomach, uterus and Medulloblastoma.

Additional cancers which can be treated by the disclosed composition according to the invention include but not limited to, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, cervical cancer, endometrial cancer, adrenal cortical cancer, and prostate cancer.

As used herein, “variant” of polypeptides refers to an amino acid sequence that is altered by one or more amino acid residues. The variant may have “conservative” changes, wherein a substituted amino acid has similar structural or chemical properties (e.g., replacement of leucine with isoleucine). More rarely, a variant may have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological activity may be found using computer programs well known in the art, for example, LASERGENE software (DNASTAR).

The term “variant,” when used in the context of a polynucleotide sequence, may encompass a polynucleotide sequence related to a wild type gene. This definition may also include, for example, “allelic”, “splice,” “species,” or “polymorphic” variants. A splice variant may have significant identity to a reference molecule, but will generally have a greater or lesser number of polynucleotides due to alternate splicing of exons during MRNA processing. The corresponding polypeptide may possess additional functional domains or an absence of domains. Species variants are polynucleotide sequences that vary from one species to another. Of particular utility in the invention are variants of wild type target gene products. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence.

The resulting polypeptides generally will have significant amino acid identity relative to each other. A polymorphic variant is a variation in the polynucleotide sequence of a particular gene between individuals of a given species. Polymorphic variants also may encompass “single nucleotide polymorphisms” (SNPs,) or single base mutations in which the polynucleotide sequence varies by one base. The presence of SNPs may be indicative of, for example, a certain population with a propensity for a disease state, that is susceptibility versus resistance.

“Diagnostic” or “diagnosed” means identifying the presence or nature of a pathologic condition or a patient susceptible to a disease. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

The terms “patient” or “individual” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters; and primates.

As used herein, a “pharmaceutically acceptable” component is one that is suitable for use with humans and/or animals without undue adverse side effects (such as toxicity, irritation, and allergic response) commensurate with a reasonable benefit/risk ratio.

As used herein, the term “safe and effective amount” refers to the quantity of a component which is sufficient to yield a desired therapeutic response without undue adverse side effects (such as toxicity, irritation, or allergic response) commensurate with a reasonable benefit/risk ratio when used in the manner of this invention. By “therapeutically effective amount” is meant an amount of a compound of the present invention effective to yield the desired therapeutic response. For example, an amount effective to delay the growth of or to cause a cancer, either a sarcoma or lymphoma, or to shrink the cancer or prevent metastasis, or to kill a virally infected cell. The specific safe and effective amount or therapeutically effective amount will vary with such factors as the particular condition being treated, the physical condition of the patient, the type of mammal or animal being treated, the duration of the treatment, the nature of concurrent therapy (if any), and the specific formulations employed and the structure of the compounds or its derivatives.

“Treatment” is an intervention performed with the intention of preventing the development or altering the pathology or symptoms of a disorder. Accordingly, “treatment” refers to both therapeutic treatment and prophylactic or preventative measures. “Treatment” may also be specified as palliative care. Those in need of treatment include those already with the disorder as well as those in which the disorder is to be prevented. In tumor (e.g., cancer) treatment, a therapeutic agent may directly decrease the pathology of tumor cells, or render the tumor cells more susceptible to treatment by other therapeutic agents, e.g., radiation and/or chemotherapy.

The “treatment of cancer”, refers to an amount of the composition, vectors and/or peptides, described throughout the specification and in the Examples which follow, capable of invoking one or more of the following effects: (1) inhibition, to some extent, of tumor growth, including, (i) slowing down and (ii) complete growth arrest; (2) reduction in the number of tumor cells; (3) maintaining tumor size; (4) reduction in tumor size; (5) inhibition, including (i) reduction, (ii) slowing down or (iii) complete prevention of tumor cell infiltration into peripheral organs; (6) inhibition, including (i) reduction, (ii) slowing down or (iii) complete prevention of metastasis; (7) enhancement of anti-tumor immune response, which may result in (i) maintaining tumor size, (ii) reducing tumor size, (iii) slowing the growth of a tumor, (iv) reducing, slowing or preventing invasion or (v) reducing, slowing or preventing metastasis; and/or (8) relief, to some extent, of one or more symptoms associated with the disorder.

By the term “modulate,” it is meant that any of the mentioned activities, are, e.g., increased, enhanced, increased, agonized (acts as an agonist), promoted, decreased, reduced, suppressed blocked, or antagonized (acts as an agonist). Modulation can increase activity more than 1-fold, 2-fold, 3-fold, 5-fold, 10-fold, 100-fold, etc., over baseline values. Modulation can also decrease its activity below baseline values.

“Cells of the immune system” or “immune cells” as used herein, is meant to include any cells of the immune system that may be assayed, including, but not limited to, B lymphocytes, also called B cells, T lymphocytes, also called T cells, natural killer (NK) cells, natural killer T (NK) cells, lymphokine-activated killer (LAK) cells, monocytes, macrophages, neutrophils, granulocytes, mast cells, platelets, Langerhans cells, stem cells, dendritic cells, peripheral blood mononuclear cells, tumor-infiltrating (TIL) cells, gene modified immune cells including hybridomas, drug modified immune cells, and derivatives, precursors or progenitors of the above cell types.

“Activity”, “activation” or “augmentation” is the ability of immune cells to respond and exhibit, on a measurable level, an immune function. Measuring the degree of activation refers to a quantitative assessment of the capacity of immune cells to express enhanced activity when further stimulated as a result of prior activation. The enhanced capacity may result from biochemical changes occurring during the activation process that allow the immune cells to be stimulated to activity in response to low doses of stimulants.

“Immune cell activity” as used herein refers to the activation of any immune cell. Activity that may be measured include, but is not limited to, (1) cell proliferation by measuring the DNA replication; (2) enhanced cytokine production, including specific measurements for cytokines, such as IFN-γ, GM-CSF, or TNF-α; (3) cell mediated target killing or lysis; (4) cell differentiation; (5) immunoglobulin production; (6) phenotypic changes; (7) production of chemotactic factors or chemotaxis, meaning the ability to respond to a chemotactin with chemotaxis; (8) immunosuppression, by inhibition of the activity of some other immune cell type; and, (9) apoptosis, which refers to fragmentation of activated immune cells under certain circumstances, as an indication of abnormal activation.

Chimeric Fusion Molecules

In a preferred embodiment, the chimeric fusion molecule binds to both an immune cell and a target tumor antigen. For example, the immune cell binding domain is a ligand for a receptor on a specific immune cell such as an NK cell, a T-cell, B-cell and the like. The target tumor antigen binding domain can be derived from a polyclonal or monoclonal antibody specific for a tumor antigen. The binding of the chimeric molecule to the immune cell targets the immune cell to the tumor expressing the tumor antigen for which the tumor antigen binding domain is specific for. Alternatively, the molecule is bound to a specific tumor via the tumor binding domain and an immune cell binds the immune cell binding domain. As such, the immune cell is activated and destruction of the tumor ensues.

As an illustrative example, not meant to limit or construe the invention in any way, we have used the targeting capabilities of an antibody to direct delivery of NKG2D ligand to the surface of tumor cells through the design and synthesis of an antibody- NKG2D ligand fusion protein. Antibody-NKG2D ligand fusion proteins can be used to treat malignancies by substituting other tumor antigenic specificities in the antibody domain (e.g. EGFR, CD20, PSMA, etc). Preferably, the murine NKG2G ligand in the antibody fusion molecule is replaced with human NKG2D ligands such the MHC class I-related chain A and B, and UL16 binding proteins (ULBP1, ULBP2, ULBP3, ULBP4) for testing in humans. Local delivery and expression of NKG2D ligands on tumor cells effectively restores the balance of NK cell activation status in favor of stimulatory signals, provides a potent costimulatory signal to CD8⁺ T cells and stimulates an effective anti-tumor response.

NKG2D ligands are inducible stress response molecules expressed on virally infected and transformed cells. NKG2D ligands activate the NKG2D receptor, a C type lectin-like receptor expressed on effector cells belonging to the innate and adaptive immune systems, and offer an effective link between innate and adaptive immunity necessary to mount potent anti-tumor response. Over-expression of NKG2D ligands has led to tumor regression in multiple murine tumor models. In contrast to observations derived from murine tumor models, the wide spread expression of these ligands on many human cancers does not generate the anticipated tumor-specific innate or adaptive response seen in mouse tumor models. One explanation for this is the shedding of these ligands into the blood stream and down-regulation of the NKG2D receptor on effector cells. This has the effect of both reducing the surface expression of these ligands on the surface of tumor cells while blunting the effectiveness of the receptor itself. Over-expression of NKG2D ligands on the surface of tumor cells effectively restores the balance of NK cell activation status in favor of stimulatory signals, provides a potent costimulatory signal to CD8⁺ T cells and can stimulate an effective anti-tumor response. Since most women who succumb to breast cancer harbor metastatic disease, direct transduction strategies effectively employed in murine experimental models to express NKG2D ligands will not be practical.

In a preferred embodiment, the compositions of the invention localize, for example, NKG2D ligands, in high concentration to the surface of cancer cells in metastatic deposits.

To produce a more effective form of NKG2D ligand, Rae-1β, we have constructed anti-HER2 IgG3-hinge-Rae-1β and anti-HER2 IgG3-C_{H}3-Rae-1β fusion proteins to explore the possibility the antibody-Rae1β fusion protein would target tumor expressing HER2 while retaining NK cell activating activity.

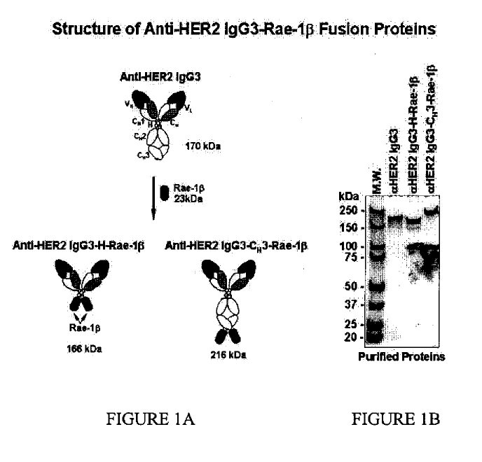

The anti-HER2 IgG3-Rae-1β genes were constructed and transfected into the murine P3X63Ag8.653 myeloma cell line. The anti-HER2 IgG3-Rae-1β fusion protein was purified using a Protein A column. An anti-HER2 IgG3-Rae-1β fusion protein of the expected molecular weight was secreted as the fully assembled H₂L₂ form (FIGS. 1A, 1B).

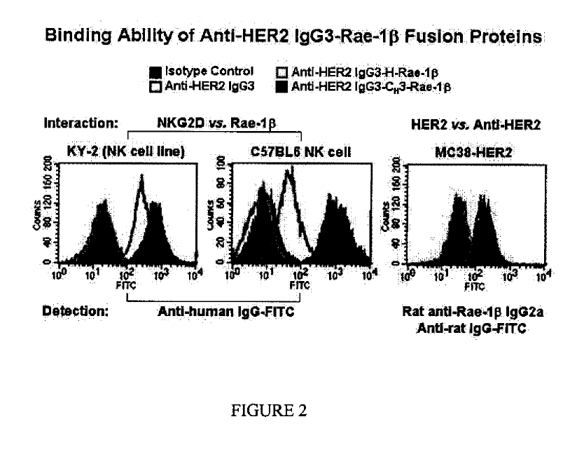

To investigate binding ability of the Rae-1β moiety in fusion proteins to NKG2D receptor using flow cytometry, anti-HER2 antibody-Rae-1β fusion proteins have been tested for binding to NK cells freshly isolated from C57BL6 or KY-2 cells (murine NK cell line) which express NKG2D (FIG. 2).

Bound anti-HER2 IgG3-Rae-1β fusion proteins to NKG2D have been detected by anti-human IgG conjugated with FITC. Anti-HER2 IgG3-C_{H}3-Rae-1β showed stronger binding ability than anti-HER2 IgG3-H-Rae-1β on both NK cells. It may be the result of conformational difference between C_{H}3-Rae-1β and H-Rae-1β, and/or due to lack of Fc region in H-Rae-1β the detection antibody, anti-human IgG-FITC, might recognize H-Rae-1β less efficiently than C_{H}3-Rae-1β. However the control antibodies, anti-dansyl IgG3 and anti-HER2 IgG3, did not show any binding to NK cells.

Whether anti-HER2 IgG3-Rae-1β fusion proteins also retained the specific binding ability to HER2 antigens has been examined with tumor cell line expressing HER2 (MC38-HER2). Bound anti-HER2 IgG3-Rae-1β fusion proteins to HER2 have been detected by anti-murine Rae-1β antibody conjugated with FITC (FIG. 2). C_{H}3-Rae-1β and H-Rae-1β showed equivalent binding ability to tumor cells expressing HER2 , while the control antibodies were not detected with anti-Rae-1β antibody-FITC.

These results demonstrate the anti-HER2 IgG3-Rae-1β fusion proteins will bind tumor cells and Rae-1β fusion proteins will bind NKG2D on NK cells through Rae-1β moiety. The NKG2D:Rae-1β interaction may stimulate NK cells and will cause tumor lysis by secreted perforin or granzyme B from the activated NK cells.

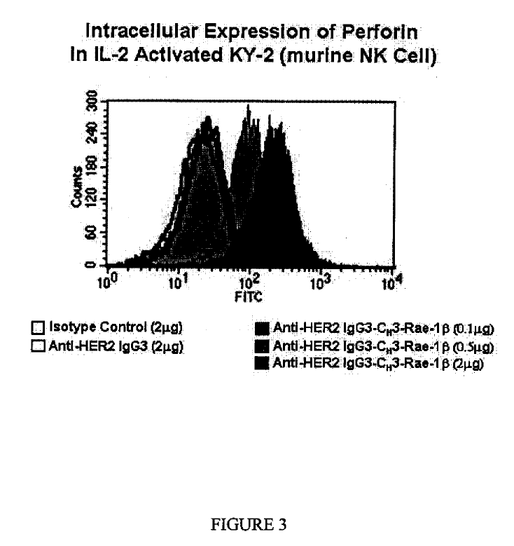

To evaluate the capacity of anti-HER2 IgG3-Rae-1β fusion protein to stimulate expression of perforin in NK cells, murine NK KY-2 cells activated with IL-2 (100U) have been stimulated in the presence of anti-HER2 IgG3-C_{H}3-Rae-1β fusion protein at the various concentrations (0.1 μg, 0.5 μg, or 2 μg and controls: anti-HER2 IgG3 (2 μg) and isotype control (2μg). Anti-HER2 IgG3-C_{H}3-Rae-1β fusion protein promote perforin expression in KY-2 cells in a dose-dependent manner (FIG. 3). This result confirmed the Rae-1β moiety of anti-HER2 IgG3-CH3-Rae-1β fusion protein is functionally correct.

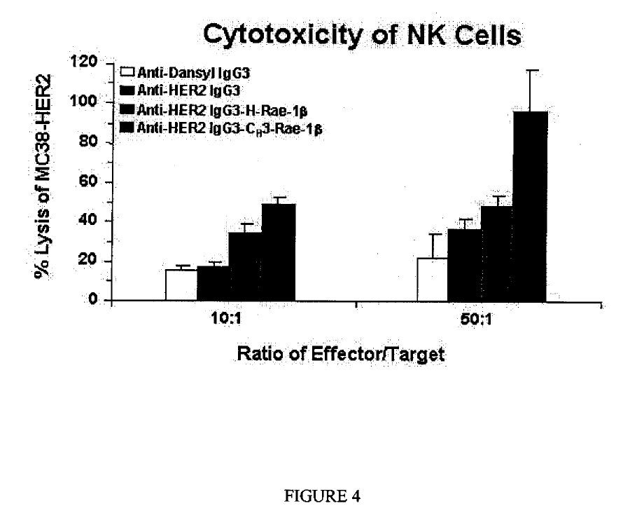

To determine whether anti-HER2 IgG3-Rae-1β fusion proteins enhance the tumoricidal activity of NK cells, freshly isolated NK cells were cultured in the presence of anti-HER2 IgG3-Rae-1β fusion proteins (10 μg/well), anti-HER2 IgG3 (10 μg/well), or control anti-dansyl IgG3 (10 μg/well). After two days of stimulation, cytotoxic potential of NK cells toward the tumor cell line, MC38 expression HER2 antigens (MC38-HER2 ), was evaluated in a 5-h ⁵¹Cr release assay (FIG. 4).

Anti-HER2 IgG3 exhibited a little of tumor-directed cytotoxicity by NK cells, while anti-dansyl IgG3 showed little cytotoxicity, suggesting the FcγRIII of NK cells is necessary for ADCC (FIG. 4). Interestingly, whereas the H-Rae-1β fusion protein exhibited only little improvement of tumor-directed cytotoxicity by NK cells, the C_{H}3-Rae-1β fusion markedly enhanced NK cell-mediated killing activity (FIG. 4). Due to lack of Fc region in the H-Rae-1β fusion protein, the cytotoxic activity of the H-Rae-1β fusion protein was less potent than the C_{H}3-Rae-1β fusion protein. These data illustrate that both Rae-1β moiety and Fc region of the fusion antibody play important roles in tumor-directed cytotoxicity mediated by NK cells.

The NKG2D binding moieties may be natural NKG2D ligands (e.g. H-60, Rae1 proteins, ULBP and MIC proteins, such as Rae1α, Rae1β and Rae1γ, particularly natural human MICA, MICB, ULBP1, ULBP2 and ULBP3 proteins), or fragments thereof, so long as the requisite binding is retained. Other preferred receptors include but not limiting to cytotoxic activating receptors such as NCR or receptors similar to NCR found on the surface of NK and T cells.

In another preferred embodiment, the compositions of the invention are used to treat pathogenic cells and diseases caused by pathogenic agents. The population of pathogenic cells may also be an exogenous pathogen or a cell population harboring an exogenous pathogen, e.g., a virus. The present invention is applicable to such exogenous pathogens as bacteria, fungi, viruses, mycoplasma, and parasites. Especially preferred are cancer causing viruses. These can be DNA or RNA viruses. Examples of DNA and RNA viruses, including, but not limited to, DNA viruses such as papilloma viruses, parvoviruses, adenoviruses, herpesviruses and vaccinia viruses, and RNA viruses, such as arenaviruses, coronaviruses, rhinoviruses, respiratory syncytial viruses, influenza viruses, picomaviruses, paramyxoviruses, reoviruses, retroviruses, and rhabdoviruses. The chimeric fusion conjugates of the invention may also be directed to a cell population harboring endogenous pathogens wherein pathogen-specific antigens are preferentially expressed on the surface of cells harboring the pathogens, and act as receptors for the ligand with the ligand specifically binding to the antigen.

The method of the present invention can be used for both human clinical medicine and veterinary applications. Thus, the host animals harboring the population of pathogenic organisms and treated with chimeric compositions may be humans or, in the case of veterinary applications, may be a laboratory, agricultural, domestic, or wild animals. The present invention can be applied to host animals including, but not limited to, humans, laboratory animals such rodents (e.g., mice, rats, hamsters, etc.), rabbits, monkeys, chimpanzees, domestic animals such as dogs, cats, and rabbits, agricultural animals such as cows, horses, pigs, sheep, goats, and wild animals in captivity such as bears, pandas, lions, tigers, leopards, elephants, zebras, giraffes, gorillas, dolphins, and whales.

The chimeric compositions is preferably administered to the host animal parenterally, e.g., intradermally, subcutaneously, intramuscularly, intraperitoneally, or intravenously. Alternatively, the conjugate may be administered to the host animal by other medically useful processes, and any effective dose and suitable therapeutic dosage form, including prolonged release dosage forms, can be used. The method of the present invention may be used in combination with surgical removal of a tumor, radiation therapy, chemotherapy, or biological therapies such as other immunotherapies including, but not limited to, monoclonal antibody therapy, treatment with immunomodulatory agents, adoptive transfer of immune effector cells, treatment with hematopoietic growth factors, cytokines and vaccination.

Other Tumor Antigens

In other preferred embodiments, the chimeric fusion molecules comprise antibodies directed at leukemia and/or lymphoma antigens, e.g. anti CD20, anti-CD22, or anti-CD52, or antibody sequences directed against lung or colon cancer antigens e.g. anti-EGFR, or prostate cancer antigens e.g. PSMA, as examples of alternative specificity. Human NKG2D ligand sequences may be substituted for murine sequences and include sequences from MICA, MICB, ULBPs or any other NKG2D ligands, NCR or cytotoxic activating receptors present on the cell surface of NK and T cells. Preferably, the chimeric fusion molecules are directed to antigens on the surface of cells, e.g. EGFR, CD20, her2/neu, GD2, GD3, IGF receptors, her2, and the like.

In accordance with the invention tumor target cells are selectively targeted by the compositions by, for example, inclusion of antibodies specific for an antigen. Tumor antigens can be the result of infection by a tumor causing virus and the viral antigens expressed on the surface of an infected cell could be targeted using this technology. Non-limiting examples of tumor antigens, include, tumor antigens resulting from mutations, such as: alpha-actinin-4 (lung carcinoma); BCR-ABL fusion protein (b3a2) (chronic myeloid leukemia); CASP-8 (head and neck squamous cell carcinoma); beta-catenin (melanoma); Cdc27 (melanoma); CDK4 (melanoma); dek-can fusion protein (myeloid leukemia); Elongation factor 2 (lung squamous carcinoa); ETV6-AML1 fusion protein (acute lymphoblastic leukemia); LDLR-fucosyltransferaseAS fusion protein (melanoma); localization or overexpression of HLA-A2^{d} (renal cell carcinoma); hsp70-2 (renal cell carcinoma); KIAA0205 (bladder tumor); MART2 (melanoma); MUM-lf (melanoma); MUM-2 (melanoma); MUM-3 (melanoma); neo-PAP (melanoma); Myosin class I (melanoma); OS-9g (melanoma); pml-RARalpha fusion protein (promyelocytic leukemia); PTPRK (melanoma); K-ras (pancreatic adenocarcinoma); N-ras (melanoma). Examples of differentiation tumor antigens include, but not limited to: CEA (gut carcinoma); gp100 /Pme117 (melanoma); Kallikrein 4 (prostate); manunaglobin-A (breast cancer); Melan-A / MART-1 (melanoma); PSA (prostate carcinoma); TRP-1/gp75 (melanoma); TRP-2 (melanoma); tyrosinase (melanoma). Over or under-expressed tumor antigens include but are not limited to: CPSF (ubiquitous); EphA3; G250/MN/CAIX (stomach, liver, pancreas); HER-2/neu; Intestinal carboxyl esterase (liver, intestine, kidney); alpha-foetoprotein (liver ); M-CSF (liver, kidney); MUCl (glandular epithelia); p53 (ubiquitous); PRAME (testis, ovary, endometrium, adrenals); PSMA (prostate, CNS, liver); RAGE-1 (retina); RU2AS (testis, kidney, bladder); survivin (ubiquitous); Telomerase (testis, thymus, bone marrow, lymph nodes); WT1 (testis, ovary, bone marrow, spleen); CA125 (ovarian).

In another preferred embodiment, abnormal or cancer cells are targeted by the compositions. For example, many malignancies are associated with the presence of foreign DNA, e.g. Bcr-Abl, Bc1-2, HPV, and these provide unique molecular targets e.g. antigens, to permit selective malignant cell targeting. The approach can be used to target expression products as a result of single base substitutions (e.g. K-ras, p53) or methylation changes. However, proliferation of cancer cells may also be caused by previously unexpressed gene products. These gene sequences can be targeted, thereby, inhibiting further expression and ultimate death of the cancer cell. In other instances, transposons can be the cause of such deregulation and transposon sequences can be targeted, e.g. Tn5.

The invention in general provides a method for treating diseases, such as cancer and diseases which are caused by infectious agents such as viruses, bacteria, intra- and extra-cellular parasites, insertion elements, fungal infections, etc., which may also cause expression of gene products by a normally unexpressed gene, abnormal expression of a normally expressed gene or expression of an abnormal gene.

The methods of the invention are preferably employed for treatment or prophylaxis against diseases caused abnormal cell growth and by infectious agents, particularly for treatment of infections as may occur in tissue such as lung, heart, liver, prostate, brain, testes, stomach, intestine, bowel, spinal cord, sinuses, urinary tract or ovaries of a subject.

In another preferred embodiment, the compositions of the invention can be administered in conjunction with chemotherapy. These chemotherapeutic agents can be co-administered, preceded, or administered after the compositions. Non-limiting examples of chemotherapeutic agents include, but not limited to: cyclophosphamide (CTX, 25 mg/kg/day,p.o), taxanes (paclitaxel or docetaxel), busulfan, cisplatin, cyclophosphamide, methotrexate, daunorubicin, doxorubicin, melphalan, cladribine, vincristine, vinblastine, and chlorambucil.

In another preferred embodiment, the pharmaceutical composition, inhibits the tumor cell growth in a subject, and the method comprises administering to the subject a pharmaceutical composition comprising a therapeutically effective amount of the composition. Inhibition of tumor cell growth refers to one or more of the following effects: (1) inhibition, to some extent, of tumor growth, including, (i) slowing down and (ii) complete growth arrest; (2) reduction in the number of tumor cells; (3) maintaining tumor size; (4) reduction in tumor size; (5) inhibition, including (i) reduction, (ii) slowing down or (iii) complete prevention, of tumor cell infiltration into peripheral organs; (6) inhibition, including (i) reduction, (ii) slowing down or (iii) complete prevention, of metastasis; (7) enhancement of anti-tumor immune response, which may result in (i) maintaining tumor size, (ii) reducing tumor size, (iii) slowing the growth of a tumor, (iv) reducing, slowing or preventing invasion and/or (8) relief, to some extent, of the severity or number of one or more symptoms associated with the disorder.

In another preferred embodiment, the compositions of the invention can be administered with immune activator compounds such as adjuvants, cytokines, other antibodies and the like. For example, compounds may be used to activate dendritic cells. Dendritic cells are highly potent APCs (Banchereau and Steinman, Nature 392:245-251, 1998) and have been shown to be effective as a physiological adjuvant for eliciting prophylactic or therapeutic antitumor immunity (see Timmerman and Levy, Ann. Rev. Med. 50:507-529, 1999). In general, dendritic cells may be identified based on their typical shape (stellate in situ, with marked cytoplasmic processes (dendrites) visible in vitro), their ability to take up, process and present antigens with high efficiency and their ability to activate naive T cell responses. Dendritic cells may, of course, be engineered to express specific cell-surface receptors or ligands that are not commonly found on dendritic cells in vivo or ex vivo, and such modified dendritic cells are contemplated by the present invention. As an alternative to dendritic cells, secreted vesicles antigen-loaded dendritic cells (called exosomes) may be used within a vaccine (see Zitvogel et al., Nature Med. 4:594-600, 1998).

Dendritic cells and progenitors may be obtained from peripheral blood, bone marrow, tumor-infiltrating cells, peritumoral tissues-infiltrating cells, lymph nodes, spleen, skin, umbilical cord blood or any other suitable tissue or fluid. For example, dendritic cells may be differentiated ex vivo by adding a combination of cytokines such as GM-CSF, IL-4, IL-13 and/or TNFα to cultures of monocytes harvested from peripheral blood. Alternatively, CD34 positive cells harvested from peripheral blood, umbilical cord blood or bone marrow may be differentiated into dendritic cells by adding to the culture medium combinations of GM-CSF, IL-3, TNFα, CD40 ligand, LPS, flt3 ligand and/or other compound(s) that induce differentiation, maturation and proliferation of dendritic cells.

Dendritic cells are conveniently categorized as “immature” and “mature” cells, which allows a simple way to discriminate between two well characterized phenotypes. However, this nomenclature should not be construed to exclude all possible intermediate stages of differentiation. Immature dendritic cells are characterized as APC with a high capacity for antigen uptake and processing, which correlates with the high expression of Fcγ receptor and mannose receptor. The mature phenotype is typically characterized by a lower expression of these markers, but a high expression of cell surface molecules responsible for T cell activation such as class 1 and class 11 MHC, adhesion molecules (e.g., CD54 and CD11) and costimulatory molecules (e.g., CD40, CD80, CD86 and 4-1 BB).

Other compounds that can be used in conjunction with the present compositions are immunostimulatory molecules. Several polynucleotides have been demonstrated to have immunostimulatory properties. For example, poly (I,C) is an inducer of interferon (IFN) production, macrophage activation and NK cell activation (Talmadge, J. E., et al. 1985. Cancer Res. 45:1058; Wiltrout, R. H. et al 1985. J. Biol. Resp. Mod. 4:512), poly (dG,dC) is mitogenic for B cells (Messina, J. P. et at. 1993. Cell. Immunol. 147:148) and induces IFN and NK activity (Tocunaga, T., Yamamoto, S., Namba, K. 1988. Jpn. J. Cancer Res. 79:682).

The method of the invention can be performed by administering to the host, in addition to the chimeric fusion compositions, compounds or compositions capable of stimulating an endogenous immune response including, but not limited to, cytokines or immune cell growth factors such as interleukins 1-18, stem cell factor, basic FGF, EGF, G-CSF, GM-CSF, FLK-2 ligand, HILDA, MIP-1α, TGF α, TGF β, M-CSF, IFN α, IFNβ, IFNγ, soluble CD23, LIF, and combinations thereof.

Therapeutically effective combinations of these cytokines may also be used. In a preferred embodiment, for example, therapeutically effective amounts of IL-2, for example, in amounts ranging from about 5000 IU/dose/ day to about 500,000 IU/dose/day in a multiple dose daily regimen, and IFN-α, for example, in amounts ranging from about 7500 IU/dose/day to about 150,000 IU/dose/day in a multiple dose daily regimen, can be used in conjunction with the compositions of the invention. In an alternate preferred embodiment IL-2, IFN-α or IFN-γ, and GM-CSF are used in combination. Preferably, the therapeutic factor(s) used, such as IL-2, IL-12, IL-15, IFN-α, IFN-γ, and GM-CSF, including combinations thereof, activate(s) natural killer cells and/or T cells. Alternatively, the therapeutic factor or combinations thereof, including an interleukin in combination with an interferon and GM-CSF, may activate other immune effector cells such as macrophages, B cells, neutrophils, LAK cells or the like. The invention also contemplates the use of any other effective combination of cytokines including combinations of other interleukins and interferons and colony stimulating factors.

In other preferred embodiments, the chimeric fusion compositions can be administered in conjunction with a chemotherapeutic agent. A “chemotherapeutic agent” is a chemical compound useful in the treatment of cancer. Examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carmomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogernanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (TAXOL®, Bristol-Myers Squibb Oncology, Princeton, N.J.) and docetaxel (TAXOTERE®, Rhône-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

Natural Cytotoxicity Receptor (NCR) Ligand Molecules

In another preferred embodiment, the invention provides chimeric molecules that include both an NCR ligand domain and carrier domain. The NCR ligand domain enhances tumor cytotoxicity (e.g., by activating NK cells), while the carrier domain confers a functional attribute to the chimeric molecule. The chimeric fusion molecule construct described herein may comprise further receptor or ligand function(s), and may comprise immunomodulating effector molecule or a fragment thereof

Humanized Antibodies

In an preferred embodiment, antibodies of the invention comprise humanized antibodies. Humanized antibodies are antibodies whose light and heavy chain genes have been constructed, typically by genetic engineering, from immunoglobulin variable and constant region genes belonging to different species. For example, the variable segments of the genes from a mouse monoclonal antibody may be joined to human constant segments, such as gamma 1 and gamma 3. A typical therapeutic chimeric antibody is thus a hybrid protein composed of the variable or antigen-binding domain from a mouse antibody and the constant or effector domain from a human antibody, although other mammalian species may be used.

As used herein, the term “humanized” immunoglobulin refers to an immunoglobulin comprising a human framework region and one or more CDR's from a non-human (usually a mouse or rat) immunoglobulin. The non-human immunoglobulin providing the CDR's is called the “donor” and the human immunoglobulin providing the framework is called the “acceptor.” Constant regions need not be present, but if they are, they must be substantially identical to human immunoglobulin constant regions, i.e., at least about 85-90%, preferably about 95% or more identical. Hence, all parts of a humanized immunoglobulin, except possibly the CDR's, are substantially identical to corresponding parts of natural human immunoglobulin sequences. A “humanized antibody” is an antibody comprising a humanized light chain and a humanized heavy chain immunoglobulin, e.g., the entire variable region of a chimeric antibody is non-human. One says that the donor antibody has been “humanized”, by the process of “humanization”, because the resultant humanized antibody is expected to bind to the same antigen as the donor antibody that provides the CDR's.

It is understood that the humanized antibodies may have additional conservative amino acid substitutions which have substantially no effect on antigen binding or other immunoglobulin functions. By conservative substitutions are intended combinations such as gly, ala; val, ile, leu; asp, glu; asn, gin; ser, thr; lys, arg; and phe, tyr.

Humanized immunoglobulins, including humanized antibodies, have been constructed by means of genetic engineering. Most humanized immunoglobulins that have been previously described have comprised a framework that is identical to the framework of a particular human immunoglobulin chain, the acceptor, and three CDR's from a non-human donor immunoglobulin chain.

A principle is that as acceptor, a framework is used from a particular human immunoglobulin that is unusually homologous to the donor immunoglobulin to be humanized, or use a consensus framework from many human antibodies. For example, comparison of the sequence of a mouse heavy (or light) chain variable region against human heavy (or light) variable regions in a data bank (for example, the National Biomedical Research Foundation Protein Identification Resource) shows that the extent of homology to different human regions varies greatly, typically from about 40% to about 60-70%. By choosing as the acceptor immunoglobulin one of the human heavy (respectively light) chain variable regions that is most homologous to the heavy (respectively light) chain variable region of the donor immunoglobulin, fewer amino acids will be changed in going from the donor immunoglobulin to the humanized immunoglobulin. Hence, and again without intending to be bound by theory, it is believed that there is a smaller chance of changing an amino acid near the CDR's that distorts their conformation. Moreover, the precise overall shape of a humanized antibody comprising the humanized immunoglobulin chain may more closely resemble the shape of the donor antibody, also reducing the chance of distorting the CDR's.

Humanized antibodies generally have advantages over mouse or in some cases chimeric antibodies for use in human therapy: because the effector portion is human, it may interact better with the other parts of the human immune system (e.g., destroy the target cells more efficiently by complement-dependent cytotoxicity (CDC) or antibody-dependent cellular cytotoxicity (ADCC)); the human immune system should not recognize the framework or constant region of the humanized antibody as foreign, and therefore the antibody response against such an antibody should be less than against a totally foreign mouse antibody or a partially foreign chimeric antibody.

Antibodies can also be genetically engineered. Particularly preferred are humanized immunoglobulins that are produced by expressing recombinant DNA segments encoding the heavy and light chain CDR's from a donor immunoglobulin capable of binding to a desired antigen, such as the tumor antigens e.g. HER2 , attached to DNA segments encoding acceptor human framework regions.

The DNA segments typically further include an expression control DNA sequence operably linked to the humanized immunoglobulin coding sequences, including naturally-associated or heterologous promoter regions. Preferably, the expression control sequences will be eukaryotic promoter systems in vectors capable of transforming or transfecting eukaryotic host cells, but control sequences for prokaryotic hosts may also be used. Once the vector has been incorporated into the appropriate host, the host is maintained under conditions suitable for high level expression of the nucleotide sequences, and, as desired, the collection and purification of the humanized light chains, heavy chains, light/heavy chain dimers or intact antibodies, binding fragments or other immunoglobulin forms may follow (see, S. Beychok, Cells of Immunoglobulin Synthesis, Academic Press, New York, (1979), which is incorporated herein by reference).

Human constant region DNA sequences can be isolated in accordance with well known procedures from a variety of human cells, but preferably immortalized B-cells (see, Kabat op. cit. and WP87/02671). The CDR's for producing preferred immunoglobulins of the present invention will be similarly derived from monoclonal antibodies capable of binding to the predetermined antigen, such as the human T cell receptor CD3 complex, and produced by well known methods in any convenient mammalian source including, mice, rats, rabbits, or other vertebrates, capable of producing antibodies. Suitable source cells for the constant region and framework DNA sequences, and host cells for immunoglobulin expression and secretion, can be obtained from a number of sources, such as the American Type Culture Collection (“Catalogue of Cell Lines and Hybridomas,” sixth edition (1988) Rockville, Md., U.S.A., which is incorporated herein by reference).

Other “substantially homologous” modified immunoglobulins to the native sequences can be readily designed and manufactured utilizing various recombinant DNA techniques well known to those skilled in the art. For example, the framework regions can vary at the primary structure level by several amino acid substitutions, terminal and intermediate additions and deletions, and the like. Moreover, a variety of different human framework regions may be used singly or in combination as a basis for the humanized immunoglobulins of the present invention. In general, modifications of the genes may be readily accomplished by a variety of well-known techniques, such as site-directed mutagenesis (see, Gillman and Smith, Gene, 8, 81-97 (1979) and S. Roberts et al., Nature, 328, 731-734 (1987), both of which are incorporated herein by reference).

Substantially homologous immunoglobulin sequences are those which exhibit at least about 85% homology, usually at least about 90%, and preferably at least about 95% homology with a reference immunoglobulin protein.

Alternatively, polypeptide fragments comprising only a portion of the primary antibody structure may be produced, which fragments possess one or more immunoglobulin activities (e.g., complement fixation activity). These polypeptide fragments may be produced by proteolytic cleavage of intact antibodies by methods well known in the art, or by inserting stop codons at the desired locations in vectors known to those skilled in the art, using site-directed mutagenesis.

In addition to microorganisms, mammalian tissue cell culture may also be used to express and produce the polypeptides of the present invention (see, Winnacker, “From Genes to Clones,” VCH Publishers, New York, N.Y. (1987), which is incorporated herein by reference). Eukaryotic cells are actually preferred, because a number of suitable host cell lines capable of secreting intact immunoglobulins have been developed in the art, and include the CHO cell lines, various COS cell lines, HeLa cells, preferably myeloma cell lines, etc, and transformed B-cells or hybridomas. Expression vectors for these cells can include expression control sequences, such as an origin of replication, a promoter, an enhancer (Queen et al., Immunol. Rev., 89, 49-68 (1986), which is incorporated herein by reference), and necessary processing information sites, such as ribosome binding sites, RNA splice sites, polyadenylation sites, and transcriptional terminator sequences. Preferred expression control sequences are promoters derived from immunoglobulin genes, SV40, Adenovirus, cytomegalovirus, Bovine Papilloma Virus, and the like.

In general, the subject humanized antibodies are produced by obtaining nucleic acid sequences encoding the variable heavy and variable light sequences of an antibody which binds a tumor antigen, preferably HER2/neu, identifying the CDRs in the variable heavy and variable light sequences, and grafting such CDR nucleic acid sequences onto human framework nucleic acid sequences.

Preferably, the selected human framework will be one that is expected to be suitable for in vivo administration, i.e., does not exhibit immunogenicity. This can be determined, e.g., by prior experience with in vivo usage of such antibodies and by studies of amino acid sequence similarities. In the latter approach, the amino acid sequences of the framework regions of the antibody to be humanized, will be compared to those of known human framework regions, and human framework regions used for CDR grafting will be selected which comprise a size and sequence most similar to that of the parent antibody, e.g., a murine antibody which binds HER2/neu. Numerous human framework regions have been isolated and their sequences reported in the literature. See, e.g., Kabat et al., (id.). This enhances the likelihood that the resultant CDR-grafted “humanized” antibody, which contains the CDRs of the parent (e.g., murine) antibody grafted onto the selected human framework regions will significantly retain the antigen binding structure and thus the binding affinity of the parent antibody.

The following references are representative of methods and vectors suitable for expression of recombinant immunoglobulins which may be utilized in carrying out the present invention. Weidle et al., Gene, 51:21-29 (1987); Dorai et al., J Immunol., 13(12):4232-4241 (1987); De Waele et al., Eur. J. Biochem., 176:287-295 (1988); Colcher et al., Cancer.Res., 49:1738-1745 (1989); Wood et al., J. Immunol., 145(a):3011-3016 (1990); Bulens et al., Eur. J. Biochem., 195:235-242 (1991); Beggington et al., Biol Technology, 10:169 (1992); King et al., Biochem. J., 281:317-323 (1992); Page et al., Biol Technology, 9:64 (1991); King et al., Biochem. J., 290:723-729 (1993); Chaudary et al., Nature, 339:394-397 (1989); Jones et al., Nature, 321:522-525 (1986); Morrison and Oi, Adv. Immunol, 44:65-92 (1988); Benhar et al., Proc. Natl. Acad. Sci. USA, 91:12051-12055 (1994); Singer et al., J Immunol., 150:2844-2857 (1993); Cooto et al., Hybridoma, 13(3):215-219 (1994); Queen et al., Proc. Natl. Acad. Sci. USA, 86:10029-10033 (1989); Caron et al., Cancer Res., 32:6761-6767 (1992); Cotoma et al., J Immunol Meth., 152:89-109 (1992). Moreover, vectors suitable for expression of recombinant antibodies are commercially available. The vector may, e.g., be a bare nucleic acid segment, a carrier-associated nucleic acid segment, a nucleoprotein, a plasmid, a virus, a viroid, or a transposable element.

After expression, the antigen binding affinity of the resulting humanized antibody will be assayed by known methods, e.g., Scatchard analysis. In a particularly preferred embodiment, the antigen-binding affinity of the humanized antibody will be at least 50% of that of the parent antibody, e.g., anti- HER2/neu , more preferably, the affinity of the humanized antibody will be at least about 75% of that of the parent antibody, more preferably, the affinity of the humanized antibody will be at least about 100%, 150%, 200% or 500% of that of the parent antibody.

In some instances, humanized antibodies produced by grafting CDRs (from an antibody which binds, for example, a tumor antigen such as, for example, HER2/neu) onto selected human framework regions may provide humanized antibodies having the desired affinity to HER2/neu. However, it may be necessary or desirable to further modify specific residues of the selected human framework in order to enhance antigen binding. This may occur because it is believed that some framework residues are essential to or at least affect antigen binding. Preferably, those framework residues of the parent (e.g., murine) antibody which maintain or affect combining-site structures will be retained. These residues may be identified by X-ray crystallography of the parent antibody or Fab fragment, thereby identifying the three-dimensional structure of the antigen-binding site. Also, framework residues involved in antigen binding may potentially be identified based on previously reported humanized murine antibody sequences. Thus, it may be beneficial to retain such framework residues or others from the parent murine antibody to optimize, for example, HER2/neu binding. Preferably, such methodology will confer a “human-like” character to the resultant humanized antibody thus rendering it less immunogenic while retaining the interior and contacting residues which affect antigen-binding.

The present invention further embraces variants and equivalents which are substantially homologous to the humanized antibodies and antibody fragments set forth herein. These may contain, e.g., conservative substitution mutations, i.e. the substitution of one or more amino acids by similar amino acids. For example, conservative substitution refers to the substitution of an amino acid with another within the same general class, e.g., one acidic amino acid with another acidic amino acid, one basic amino acid with another basic amino acid, or one neutral amino acid by another neutral amino acid. What is intended by a conservative amino acid substitution is well known in the art.

Methods of Delivering a Chimeric Molecule to a Cell

The invention also provides a method of delivering chimeric molecule to a cell. The chimeric molecules of the invention can be delivered to a cell by any known method. For example, a composition containing the chimeric molecule can be added to cells suspended in medium. Alternatively, a chimeric molecule can be administered to an animal (e.g., by a parenteral route) having a cell expressing a receptor that binds the chimeric molecule so that the chimeric molecule binds to the cell in situ. For example, the chimeric molecules of this invention that feature an Ig domain that is specific for HER2/neu are particularly well suited as targeting moieties for binding tumor cells that overexpress HER2/neu, e.g., breast cancer and ovarian cancer cells.

Administration of Compositions to Animals

For targeting a tumor cell in situ, the compositions described above may be administered to animals including human beings in any suitable formulation. For example, compositions for targeting a tumor cell may be formulated in pharmaceutically acceptable carriers or diluents such as physiological saline or a buffered salt solution. Suitable carriers and diluents can be selected on the basis of mode and route of administration and standard pharmaceutical practice. A description of exemplary pharmaceutically acceptable carriers and diluents, as well as pharmaceutical formulations, can be found in Remington's Pharmaceutical Sciences, a standard text in this field, and in USP/NF. Other substances may be added to the compositions to stabilize and/or preserve the compositions.

The compositions of the invention may be administered to animals by any conventional technique. The compositions may be administered directly to a target site by, for example, surgical delivery to an internal or external target site, or by catheter to a site accessible by a blood vessel. Other methods of delivery, e.g., liposomal delivery or diffusion from a device impregnated with the composition, are known in the art. The compositions may be administered in a single bolus, multiple injections, or by continuous infusion (e.g., intravenously). For parenteral administration, the compositions are preferably formulated in a sterilized pyrogen-free form.

Kits

Kits according to the present invention include frozen or lyophilized chimeric molecules to be reconstituted, respectively, by thawing (optionally followed by further dilution) or by suspension in a (preferably buffered) liquid vehicle. The kits may also include buffer and/or excipient solutions (in liquid or frozen form)—or buffer and/or excipient powder preparations to be reconstituted with water—for the purpose of mixing with the chimeric molecules to produce a formulation suitable for administration. Thus, preferably the kits containing the chimeric molecules are frozen, lyophilized, pre-diluted, or pre-mixed at such a concentration that the addition of a predetermined amount of heat, of water, or of a solution provided in the kit will result in a formulation of sufficient concentration and pH as to be effective for in vivo or in vitro use in the treatment or diagnosis of cancer. Preferably, such a kit will also comprise instructions for reconstituting and using the chimeric molecule composition to treat or detect cancer. The above-noted buffers, excipients, and other component parts can be sold separately or together with the kit.

It will be recognized by one of skill in the art that the optimal quantity and spacing of individual dosages of a chimeric molecule of the invention will be determined by the nature and extent of the condition being treated, the form, route and site of administration, and the particular animal being treated, and that such optima can be determined by conventional techniques. It will also be appreciated by one of skill in the art that the optimal course of treatment, i.e., the number of doses of chimeric molecules thereof of the invention given per day for a defined number of days, can be ascertained by those skilled in the art using conventional course of treatment determination tests.

The following examples are offered by way of illustration, not by way of limitation. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

All publications and patent documents cited in this application are incorporated by reference for all purposes to the same extent as if each individual publication or patent document were so individually denoted. By their citation of various references in this document, Applicants do not admit any particular reference is “prior art” to their invention.

### EXAMPLES

### Example 1

Construction, Expression, and Characterization of anti-HER2 IgG3-NKG2D Ligand (Rae-1B) Fusion Proteins

Experimental murine Rae-1β gene originated from pCR-Blunt II-TOPO-Rae-1β by PCR using primers 5′-CCCCTCGAGCTCTGGATGATGCACACTCTCTTAGG-3′ (SEQ ID NO: 1) and 5′-CCCCGAATTCGTTAACCTTTCTTAGAAGTAGAGTGG-3′ (SEQ ID NO: 2). PCR products were subcloned into pCR-Blunt II-TOPO. The subcloned Rae-1β gene was ligated in frame to either the end of the hinge region or the carboxyl end of the heavy chain constant domain (C_{H}3) of human IgG3 in the vector pAT135 and the Rae-1β heavy chain constant region was then joined to an anti-HER2 variable region of a recombinant humanized monoclonal antibody 4D5-8 (rhuMAb HER2 , trastuzumab; Genentech, San Francisco, Calif.) in the expression vector (pSV2-his) containing HisD gene for eukaryotic selection.

The anti-HER2 IgG3-Rae-1β fusion protein constructs was stably transfected into P3X63Ag8.653 myeloma cells stably expressing the anti-HER2 κ light chain in order to assemble entire anti-HER2 IgG3-Rac-1β fusion proteins. The anti-HER2 IgG3-Rae-1β fusion proteins were biosynthetically labeled with [³⁵S]-methionine and analyzed by SDS-PAGE. The Rae-1β fusion protein was purified from culture supernatants using protein A immobilized on Sepharose 4B fast flow and analyzed by SDS-PAGE.

### Example 2

Binding analysis of anti-HER2 IgG3-Rae-1β fusion proteins.

To investigate binding ability of the Rae-1β moiety in fusion proteins to NKG2D receptor using flow cytometry, anti-HER2 antibody-Rae-1β fusion proteins (10 μg), anti-HER2 IgG3 (10 μg), and isotype control (10 μg, anti-dansyl IgG3) have been incubated with 0.2-1×10⁶ NK cells freshly isolated from C57BL6 or KY-2 cells (murine NK cell line) for 1 hour at 4° C. After washing them with PBS twice, the bound anti-HER2 IgG3-Rae-1β fusion proteins bound to NKG2D have been detected by anti-human IgG conjugated with FITC (1 hour at 4° C., 2 μl/sample). Fluorescent intensity has been analyzed with LSR flow cytometry. Whether anti-HER2 IgG3-Rae-1β fusion proteins also retained the specific binding ability to HER2 antigens has been examined with MC38 tumor cells expressing HER2 (MC38-HER2 ). Anti-HER2 antibody-Rae-1β fusion proteins (10 μg), anti-HER2 IgG3 (10 μg), and isotype control (10 μg, anti-dansyl IgG3) have been incubated with 1×10⁶ MC38-HER2 for 1 hour at 4° C. The bound anti-HER2 IgG3-Rae-1β fusion proteins to HER2 have been detected by rat anti-murine Rae-1β antibody and anti-rat antibody conjugated with FITC. Fluorescent intensity has been analyzed with LSR flow cytometry.

### Example 3

Analysis of perforin production in KY-2 with anti-HER2 IgG3-Rae-1β fusion protein.

To evaluate the capacity of anti-HER2 IgG3-Rae-1β fusion protein to stimulate expression of perforin in NK cells, the 48 well tissue culture plates were coated with MC38-HER2 (0.5×10⁶) and fixed with 2% paraformaldehyde for 30 min. After twice washing with PBS, KY-2 cells (1×10⁶, murine NK cell line) cultured with IL-2 (100U) have been treated overnight with anti-HER2 IgG3-CH3-Rae-1β fusion protein at the various concentrations (0.1 μg, 0.5 μg, or 2 μg) and controls: anti-HER2 IgG3 (2 μg) and isotype control (2 μg). To evaluate intracellular perforin expression KY-2 cells have been treated with brefeldin A (10 μg/ml) for 4 hours and the perforin expression has been assayed with anti-perforin conjugated with FITC. Fluorescent intensity has been analyzed with LSR flow cytometry.

### Example 4

Analysis of tumor-directed NK cell-mediated cytotoxicity by anti-HER2 IgG3-Rae-1β fusion protein.

To determine whether anti-HER2 IgG3-Rae-1β fusion proteins enhance the tumoricidal activity of NK cells, freshly isolated NK cells were stimulated in the presence of anti-HER2 IgG3-Rae-1β fusion proteins (10 μg/well), anti-HER2 IgG3 (10 μg/well), or control anti-dansyl IgG3 (10 μg/well). After 2 days, NK were cocultured in round-bottom 96-well plates with the ⁵¹Cr-labeled tumor cell lines MC38-HER2 (1×10⁴) at different E:T ratios (10:1 and 50:1). After 5 h of incubation, chromium release was measured. Maximal and spontaneous releases were measured by treating labeled cells with 2% Triton X-100 or medium alone, respectively. The specific cytotoxicity was calculated according to this formula: percent-specific lysis=100×((cpm experimental release−cpm spontaneous release)/(cpm maximal release−cpm spontaneous release)). The results of three different donors are presented as mean±SE of triplicate wells.

### Example 5

Development of an anti-HER2 antibody-NKG2D ligand fusion protein for breast cancer therapy

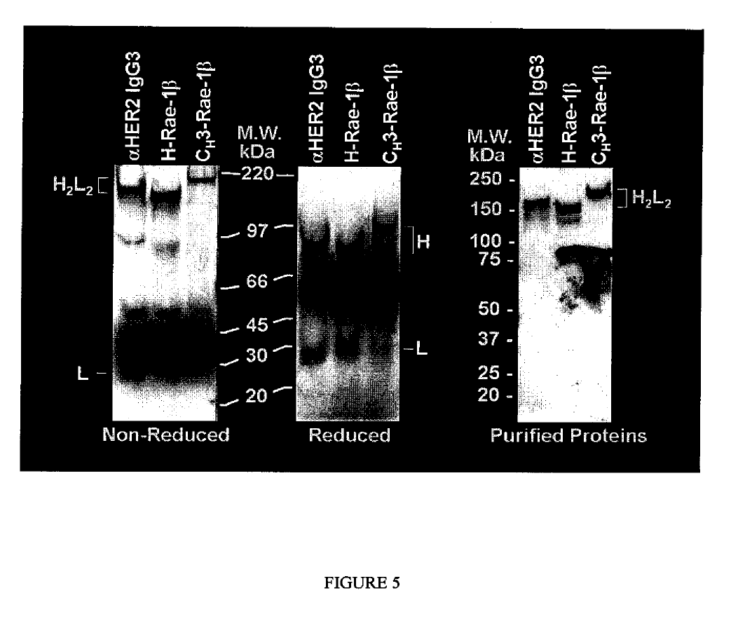

Structure of anti-HER2 IgG3-Rae-1β Fusion Proteins: Anti-HER2 IgG3-Rae-1β fusion proteins of the expected molecular weight were secreted as the fully assembled H₂L₂ form (FIG. 5).

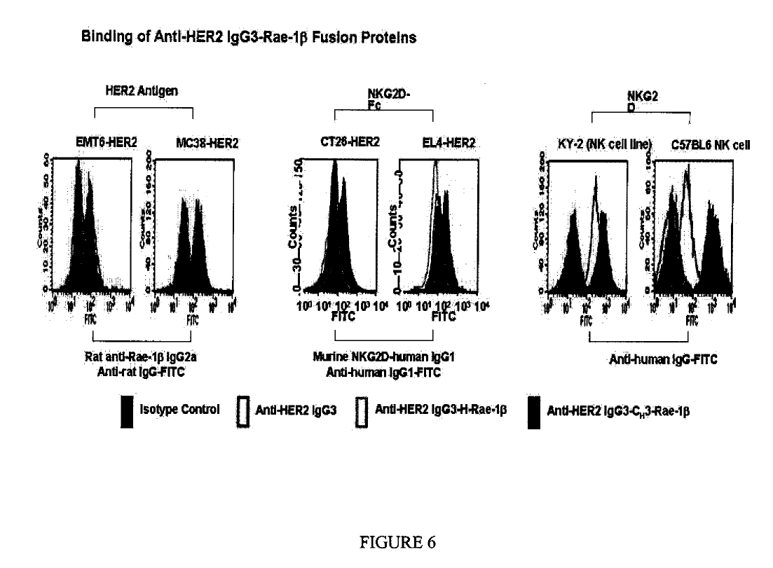

Binding of anti-HER2 IgG3-Rae-1β Fusion Proteins: Anti-HER2 IgG3-Rae-1b fusion proteins bound to HER2+ on the surface of tumor cells and Rae-1β fusion proteins recognized NKG2D receptor as displayed on the NKG2D-Fc (human IgG1) fusion protein or on NK cells through Rae-1β moiety. Hinge-Rae-1β fusion showed reduced binding to NKG2D compared to CH3-Rae-1β (FIG. 6).

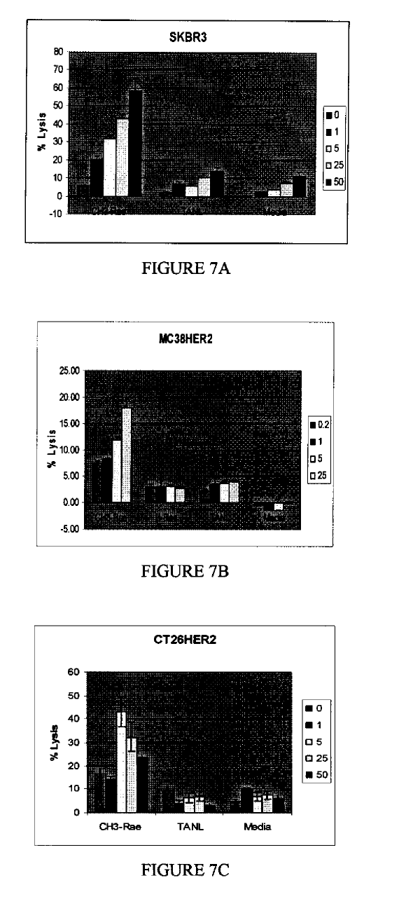

NK Cell-Mediated Direct Lysis (Murine NK Cell KY-2 ): Anti-HER2 IgG3 (IgG3) and effector cells exhibited little tumor-directed cytotoxicity (5-10%) using KY-2 cells as effectors at the indicated effector:target ratios. H-Rae-1β fusion protein exhibited very little tumor-directed cytotoxicity (5%). Incubation of targets with CH3-Rae-1β fusion markedly enhanced NK cell-mediated killing (15-59%). This result indicated that the enhanced lytic activity of KY-2 with CH3-Rae-1β, which retains an intact Fc region bound to Rae-1β, might be due to the NKG2D/Rae-1β interactions, since anti-HER2 IgG3 with Fc region showed minimal lysis (FIGS. 7A-7C).

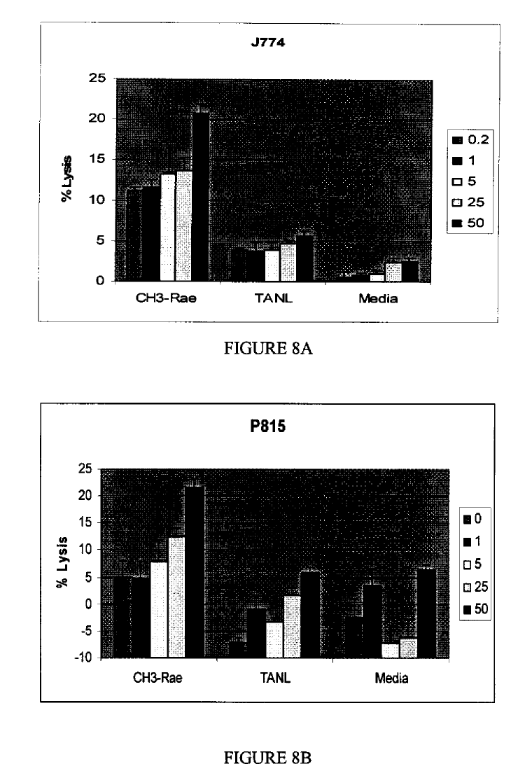

Redirected Lysis with KY2: Up to 22% redirected lysis was observed when P815 or J774 cells primed with anti-HER2 IgG3-CH3-Rae-1β were incubated with KY-2 cells. This lysis was greater than that seen with control anti-HER2 IgG3 (<6%). These results suggested that the NKG2D/Rae-1β interactions were necessary for the lytic activity of FcR+P815 or J774 cells (FIGS. 8A, 8B).

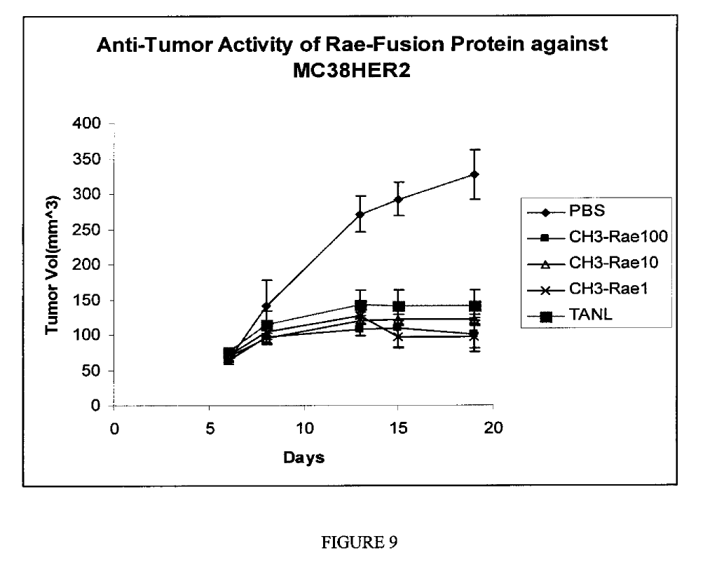

Anti-tumor Activity of anti-HER2 IgG3-CH3-Rae-1β against MC38-HER2: Anti-HER2 IgG3-Rae-1β fusion proteins inhibited the growth of the murine MC38-HER2 in C57BL6 to a greater extent than PBS. However, MC38-HER2 tumors (2/5) were spontaneously regressed in PBS group (FIG. 9).

### Other Embodiments

While the above specification contains many specifics, these should not be construed as limitations on the scope of the invention, but rather as examples of preferred embodiments thereof. Many other variations are possible. Accordingly, the scope of the invention should be determined not by the embodiments illustrated, but by the appended claims and their legal equivalents.

References

1. Diefenbach A, Jamieson AM, Liu SD, Shastri N, Raulet DH. Ligands for the murine NKG2D receptor: expression by tumor cells and activation of NK cells and macrophages. Nat Immunol. 2000;1:119-126.
2. Diefenbach A, Raulet DH. The innate immune response to tumors and its role in the induction of T-cell immunity. Immunol Rev. 2002;188:9-21.
3. Diefenbach A, Jensen ER, Jamieson AM, Raulet DH. Rae1 and H60 ligands of the NKG2D receptor stimulate tumour immunity. Nature. 2001;413:165-171.
4. Cerwenka A, Baron JL, Lanier LL. Ectopic expression of retinoic acid early inducible-1 gene (RAE-1) permits natural killer cell-mediated rejection of a MHC class I-bearing tumor in vivo. Proc Natl Acad Sci U S A. 2001;98:11521-11526.
5. Shin SU, Friden P, Moran M, Olson T, Kang YS, Pardridge WM, Morrison SL. Transferrin-antibody fusion proteins are effective in brain targeting. Proc Natl Acad Sci U S A. 1995; 92(7):2820-4.

What is claimed is:

1. A composition comprising a chimeric fusion molecule, wherein the chimeric fusion molecule comprises a tumor antigen binding domain and an immune cell binding domain.

2. The composition of claim 1, wherein the antigen binding domain comprises an isolated antibody or fragments thereof.

3. The composition of claim 2, wherein the isolated antibody comprises immunoglobulin variable and constant regions.

4. The composition of claim 2, wherein the isolated antibody or fragments thereof are fused to an immune cell binding domain.

5. The composition of claim 1 wherein the immune cell binding domain is fused via the immunoglobulin constant region; C_{H}1, hinge, C_{H}2, or C_{H}3 domain of human IgG1, IgG2, IgG3 or IgG4.

6. The composition of claim 1, wherein the immune cell binding is a ligand specific for an NK cell receptor, a monocyte receptor, a B-cell surface receptor, and/or a T cell surface receptor.

7. The composition of claim 1, wherein immune cell binding is a ligand specific for a natural killer cell receptor (NK cell).

8. The composition of claim 1, wherein the immune cell ligand is an NKG2D ligand and variants thereof and/or MHC class I alpha and beta chains and/or UL 16 binding proteins.

9. The composition of claim 1, wherein the UL16 binding proteins are selected from the group consisting of ULBP1, ULBP2, ULBP3, and ULBP4.

10. The composition of claim 1, wherein the anti-tumor antigen binding domain is a monoclonal and/or polyclonal antibody variable region.

11. The composition of claim 1, wherein a tumor antigen comprises HER2 , human telomerase reverse transcriptase (hTERT), cytochrome P450 isoform 1B1 (CYP1B1) CA 27.29, CA 15-3 antigen, or leukemia and/or lymphoma antigens, anti CD20, anti-CD22, or anti-CD52, or antibody sequences directed against lung or colon cancer antigens, anti-EGFR, or prostate cancer antigens, PSMA.

12. The composition of claim 1, wherein the chimeric molecule is comprised within a pharmaceutical carrier.

13. The composition of claim 1, wherein the chimeric fusion molecule is anti-HER2 IgG3-Rae-1β.

14. A nucleic acid expressing a chimeric fusion molecule, wherein the chimeric fusion molecule comprises a tumor antigen binding domain and an immune cell binding domain.

15. The nucleic acid of claim 14, wherein the immune cell binding domain is obtainable by polymerase chain reactions using primers SEQ ID NO's: 1 and 2.

16. The nucleic acid of claim 14, wherein the immune cell binding domain nucleic acids are ligated to nucleic acid sequences expressing an anti-tumor antigen binding domain of an antibody.

17. A method of treating cancer in an animal subject, the method comprising administering to the animal subject a pharmaceutical composition comprising a chimeric fusion molecule, wherein the chimeric fusion molecule comprises a tumor antigen binding domain and an immune cell binding domain.

18. The method of claim 17, wherein the immune cell binding domain is a ligand specific for an NK cell receptor, a monocyte receptor, a B-cell surface receptor, and/or a T cell surface receptor.

19. The method of claim 17, wherein immune cell binding is a ligand specific for a natural killer cell receptor (NK cell).

20. The method of claim 17, wherein the immune cell ligand is an NKG2D ligand and variants thereof and/or MHC class I alpha and beta chains and/or UL 16 binding proteins.

21. The method of claim 17, wherein the tumor antigen comprises HER2 , human telomerase reverse transcriptase (hTERT), cytochrome P450 isoform 1B1 (CYP1B1) CA 27.29, CA 15-3 antigen or leukemia and/or lymphoma antigens, anti CD20, anti-CD22, or anti-CD52, or antibody sequences directed against lung or colon cancer antigens, anti-EGFR, or prostate cancer antigens, PSMA.
